(19) United States
(12) Patent Application Publication
(10) Pub. No.: US 2024/0299405 A1
(43) Pub. Date: Sep. 12, 2024

(54) APPLICATION OF ATP POTASSIUM CHANNEL MODIFIER IN PREPARATION OF ANTI-DIABETIC NEPHROPATHY DRUG

(71) Applicant: HANGZHOU QIAN BIOTECHNOLOGY LTD, Zhejiang (CN)

(72) Inventors: Yuan CHEN, Zhejiang (CN); Jiang ZHU, Zhejiang (CN)

(73) Assignee: HANGZHOU QIAN BIOTECHNOLOGY LTD, Zhejiang (CN)

(21) Appl. No.: 18/013,252

(22) PCT Filed: May 28, 2021
(86) PCT No.: PCT/CN2021/096736

§ 371 Date: Jul. 7, 2023

(30) Foreign Application Priority Data
May 28, 2020 (CN) 202010471213.8

(51) Int. Cl. A61K 31/549; A61K 45/00; A61P 13/12

(52) U.S. Cl. CPC A61K 31/549; A61K 45/00; A61P 13/12

(57) ABSTRACT

A new pharmaceutical use of potassium ATP channel modulator, namely potassium ATP channel modulator (such as diazoxide, cromakalim, pinacidil, nicorandil, and aprikalim, etc.) in preparation of antidiabetic nephropathy drugs, is provided. Also provided with a potassium ATP channel modulator as an active ingredient of the pharmaceutical composition is used for the prevention or treatment of diabetic nephropathy. A diabetic rat model created by streptozotocin (STZ) is used and a diabetic nephropathy model with a high-fat diet in rats is generated. Urine microalbumin (mALB) is used as an indicator to test whether the rat enters diabetic nephropathy. In addition, rat body weight and its blood glucose level are used as an indicator to decide whether the model is successful. The experimental results showed that the administration of potassium ATP channel openers such as diazoxide to diabetic rats could delay the process of kidney injury in diabetic rats.

### BACKGROUND

### Technical Field

The present invention relates to the chemical technology field of potassium ATP (KATP) channel regulator, specifically relates to a new pharmaceutical application of a KATP channel regulator, that is, the application of potassium ATP channel modulator in the preparation of antidiabetic nephropathy drugs.

### Description of Related Art

Diabetic Kidney Disease (DKD) is one of the most important microvascular complications of diabetes, the main cause of chronic kidney disease, and the most common condition leading to End stage renal disease (ESRD). In fact, most diabetic patients who die from cardiovascular disease also have diabetic nephropathy, and the cause of death is strongly associated with diabetic nephropathy (Afkarian M, Sachs M C, Kestenbaum B, Hirsch I B, Tuttle K R, Himmelfarb J, de Boer I H. Kidney disease and increased mortality risk in type 2 diabetes[J]. Journal of the American Society of Nephrology. 2013, 24(2): 302-308.) ∘ Both patients with type 1 diabetes mellitus (TlD) and type 2 diabetes mellitus (T2D) have significant renal impairment and certain urinary albumin (Dwyer J P, Parving H H, Hunsicker L G, Ravid M, Remuzzi G, Lewis J B. Renal dysfunction in the presence of normoalbuminuria in type 2 diabetes: results from the DEMAND Study[J]. Cardiorenal Med. 2012, 2(10): 1-10.) ∘ As the number of diabetic patients increases, the number of diabetic nephropathy patients is also gradually increasing, and diabetic nephropathy has been called a “global medical disaster”. Type 2 diabetes is the leading cause of kidney disease in the United States and the fifth fastest-growing cause of death globally.

The pathological changes in diabetic nephropathy are mainly caused by prolonged hyperglycemia. The main functional unit of the kidney is the glomerulus, which consists of about 1 million glomeruli. The pathological changes are mainly manifested as initial compensatory hyperfiltration, which gradually becomes hypofiltration over time, mainly due to thickening of the glomerular basement membrane and widening of the mesangium, until the filtration of the entire glomerulus can eventually be turned off.

According to the pathophysiological characteristics and the development of diabetic nephropathy, the current academic community adopts the staging standard of “Mogensen staging” to divide diabetic nephropathy into 5 stages (ZHAO Jinxi, WANG Shidong, LI Jing, HUANG Weijun. Differentiation specification and efficacy evaluation scheme of diabetic nephropathy and its research[J]. World Medicine. 2017,12(1):1-4.):

Stage I (hyperplasia and hyperfiltration stage): the structure of the glomerulus at this stage is normal and pathological changes have not occurred, but the kidneys are enlarged, the glomerular filtration rate (GFR) increases, and the GFR can decrease after insulin therapy and the proper control of hyperglycemia.

Stage II. (preclinical): histological changes occur at this stage, pathological examination can show mild thickening of the glomerular basement membrane (GBM), and normal urinary albumin excretion rate (UAE) in the kidney (<30 mg/24 hours) (eg, at rest), or intermittent microalbuminuria (eg, after exercise, stress), but the lesion is still reversible;

Stage III. (early diabetic nephropathy): pathological examination at this stage can find thickening of the glomerular basement membrane and further widening of the mesangium, UAE is 30-300 mg/24 h, showing persistent microalbuminuria, and blood pressure is increased;

Stage IV (clinical diabetic nephropathy): pathological examination at this stage can find more severe glomerular lesions (such as glomerulosclerosis, focal tubular atrophy and interstitial fibrosis), persistent proteinuria, sustainability accompanied by hypertension, edema, dyslipidemia, and decreased GFR;

Stage V (renal insufficiency): end-stage renal failure, elevated serum creatinine, hypertension, clinical manifestations of uremia; GFR<15 or dialysis required.

Insulin resistance is associated with the development of glomerular filtration hypercompensatory hyperfiltration and is common in the initial stages of diabetic nephropathy (Mogensen C E. Early glomerular hyperfiltration in insulin-dependent diabetics and late nephropathy[J]. Scandinavian Journal of Clinical and Laboratory Investigation. 1986, 46(3): 201-206.), metabolic and hemodynamic interactions play a key role in the pathophysiological mechanisms leading to kidney disease progression (Caramori M L, Fioretto P, Mauer M. Low glomerular filtration rate in normoalbuminuric type 1 diabetic patients: an indicator of more advanced glomerular lesions[J]. Diabetes. 2003, 52(4): 1036-1040.) ∘ In most cases, proteinuria and reduced glomerular filtration rate usually occur at the same time, which means that GFR is reduced when albumin is produced, and the period of compensation has passed. Studies have shown that a small number of patients may have diabetic nephropathy without increasing the UAE (Mogensen C E. Glomerular filtration rate and renal plasma flow in short-term and long-term juvenile diabetes mellitus[J]. Scandinavian Journal of Clinical & Laboratory Investigation. 1971, 28(1): 91-100.), that is, when urine albumin values are within the normal range, GFR is already decreasing in some patients. About 10% of patients with T2D have a low GFR without microalbuminuria, and this phenomenon has also been observed in patients with T1D and nephropathy with microalbuminuria (Perkins B A, Krolewski A S. Early nephropathy in type 1 diabetes: the importance of early renal function decline[J]. Current Opinion in Nephrology and Hypertension. 2009, 18(3): 233-240). Therefore, urine microalbumin testing is an early symptom, and screening for glomerular filtration rate can help detect more severe diabetic nephropathy.

A large proportion of diabetic nephropathy patients have type 2 diabetes. But in fact, diabetic nephropathy is less likely to be in type 2 diabetes than in type 1 diabetes. There is about 20% to 25% chance to develop into diabetic nephropathy in type 2 diabetes and about 10% more chance to progress into diabetic nephropathy in type 1 diabetes than in type 2 diabetes. However, there are far more people with type 2 diabetes than with type 1 diabetes, therefore, there are far more diabetic nephropathy patients with type 2 diabetes in terms of the total. Hyperglycemia is the most important risk factor for diabetic nephropathy, and other risk factors include high blood pressure, smoking, dyslipidemia, proteinuria, glomerular hyperfiltration and dietary factors.

Treatment of prediabetic nephropathy is mainly to delay the development or progression of the disease. In patients with type 1 diabetes, nephropathy is characterized by thickening of the glomeruli and tubular basement membrane with progressive glomerular membrane expansion (diffuse or nodular), resulting in a gradual decrease in the glomerular filtration surface, along with changes in interstitial morphology and clearance of afferent and efferent glomerular arterioles. In patients with type 2 diabetes, kidney damage is heterogeneous and more complex than in individuals with type 1 diabetes. For optimal metabolic control, drugs that block the renin-angiotensin-aldosterone system are effective strategies for hypertension (<130/80 mmHg) and dyslipidemia (LDL cholesterol<100 mg/dl) to prevent the development of microalbuminuria and delay progression in patients with kidney disease.

Currently, there are two classes of drugs to treat diabetic nephropathy: ACE inhibitors and SGLT-2 inhibitors. The representative drug of ACE inhibitors is Captopril, a new drug developed by Bristol-Myers Squibb (BMS) and approved by the US FDA in October 1993 for the treatment of diabetic nephropathy complicated by type 1 diabetes. The mechanism of ACE inhibitors in the treatment of diabetic nephropathy is not clear in the academic circles, and most scholars believe that it intervenes in the renin angiotensin system, reduces the pressure of the exit of the tubular arterioles, and reduces the tubular pressure. Therefore, results in the treatment of diabetic nephropathy. The clinical manifestation is that Captopril can delay and slow the progression of albuminuria (WAN Xiaolin. Captopril in the treatment of diabetic nephropathy[J]. Herald of Medicine, 1995(03): 126). The representative drug of SGLT-2 inhibitors is canagliflozin, a new drug developed by Janssen Corporation, a subsidiary of Johnson Group, which was approved by the US FDA in March 2013 for the treatment of type 2 diabetes, and in October 2019 by the US FDA for the treatment of diabetic nephropathy complicated by type 2 diabetes, which is also the only drug approved to treat diabetic nephropathy in patients with type 2 diabetes mellitus (T2D). Hypoglycemic therapy that can also reduce the risk of hospitalization for heart failure.

ATP-sensitive potassium (K_{ATP}) channels play an important role in a variety of tissues by coupling cellular metabolism to electrical activity. K_{ATP} channels exist in various combinations of SUR and Kir subunits to form different subtypes or subclasses. SUR1 in combination with Kir6.x subunits usually forms adipocyte and pancreatic B-cell type K_{ATP} channels, while SUR2A in combination with Kir6.x and SUR2B with Kir6.x usually form cardiac-type and smooth muscle type K_{ATP} channels (Babenko A P, Aguilar-Bryan L, Bryan J. A view of sur/kir6.x, K_{ATP} channels. Annu Rev Physiol 1998; 60:667-687). These potassium channels are inhibited by intracellular ATP and activated by intracellular nucleotides diphosphate. This K_{ATP} channel links the metabolic state of the cell to the plasma membrane potential and plays a major role in regulating cell activity.

Since K_{ATP} channels can open and close channels by sensing the ratio of ADP and ATP within cells, K_{ATP} activation causes membrane hyperpolarization at rest, while its inhibition produces membrane depolarization. K_{ATP} channels can be studied by linking cellular metabolism to the electrical activity of the plasma membrane. In recent years, the role of K_{ATP} channels in glucose homeostasis and ischemia protection has been studied, and sulfonylureas have been found to lower blood sugar. In addition, some other roles of K_{ATP} channels have been discovered, for example, through the K_{ATP} channel can protect the apoptosis of nerve cells after stroke, K_{ATP} channel can also regulate male reproductive behavior, human memory is also related to K_{ATP} channel in the brain, and so on. However, the association of potassium-ATP channels with diabetic nephropathy has not been reported.

Diazoxide (Diazoxide) alias antihypertensive azine, chemical name: 7-chloro-3-methyl-2-hydro-1,2,4-benzothiadiazine 1,1-dioxide, CAS number: 364-98-7, molecular formula: C8H7ClN2O2S, structural formula is:

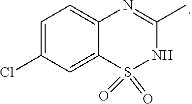

Diazoxide is a K_{ATP} channel agonist known to be used in the treatment of the following conditions: 1) hypertensive emergencies; 2) Hyperinsulin hypoglycemia; 3) Idiopathic hypoglycemia in young children. In addition, the literature reports the following regarding the amplification indication of diazoxide:

A Chinese invention patent with publication number CN 101043879A, disclosing that diazoxide can be used to treat obesity and psychosis.

U.S. Pat. No. 5,629,045 U.S. invention patent, disclosing that diazoxide can be used for topical ophthalmic application.

A Chinese invention patent with publication number CN 107106500A, disclosing that diazoxide can be used to treat Prader-Willi syndrome or Smith-Margili syndrome.

After the applicant's search, it was found that there is no literature reporting the correlation between K_{ATP} channel agonists and the treatment of diabetic nephropathy, nor has there been any literature reporting that K_{ATP} channel openers (such as diazepine, cromakalim, pinacidil, nicorandil, aprikalim, etc.) can be used for the treatment of diabetic nephropathy; In particular, the selection of specific doses of diazoxide is effective in the prevention or treatment of diabetic nephropathy in the early stages, which is not known to those skilled in the art.

### SUMMARY

The technical problem to be solved by the present invention lies in providing a new pharmaceutical use of diazepine and K_{ATP} channel agonists for the treatment of diabetic nephropathy, particularly for the early stages of diabetic nephropathy.

For this purpose, the present invention adopts the following technical solution:

The present invention provides an application of potassium ATP channel modulator in the preparation of antidiabetic nephropathy drugs.

Preferably, diabetic nephropathy is diabetic nephropathy complicated by type 1 diabetes mellitus and/or type 2 diabetes mellitus.

More preferably, the stage evolution of this diabetic nephropathy is in stage I, II or III.

Preferably, potassium ATP channel modulators include potassium ATP channel openers or potassium ATP channel inhibitors.

More preferably, potassium ATP channel modulators are selected from one of diazepine, cromakalim, pinacidil, nicorandil, aprikalim, quinethazone, minoxidil, and nicardipine.

Preferably, potassium ATP channel opener is diazoxide administered at a dose of 0.5-5 mg/kg.

The present invention also provides a pharmaceutical composition for the treatment of diabetic nephropathy, the pharmaceutical composition includes a potassium ATP channel regulator as an active ingredient as described above.

Preferably, the pharmaceutical composition includes pharmaceutically acceptable excipients.

Preferably, the pharmaceutical composition is used for the prevention or treatment of early diabetic nephropathy, specifically referring to stage I, II or III in the evolution of diabetic nephropathy stages.

Preferably, the dosage form of the pharmaceutical composition is selected from one of tablets, capsules, granules, injections, patches, gels.

Preferably, the aforementioned pharmaceutically acceptable excipients are fillers, disintegrants, binders, thinners, lubricants, regulators, solubilizers, co-solvents, emulsifiers of one or more of them.

### Definitions of Related Terms

The “pharmaceutical composition” referred to in the present invention refers to one or more compounds of the present invention or salts thereof and a carrier for delivering bioactive compounds to organisms (e.g., humans) generally accepted in the art. The purpose of the pharmaceutical composition is to facilitate the delivery of the administration of the organism.

The term “pharmaceutically acceptable carrier” means a substance co-administered with and facilitated by the active ingredient, including, without limitation, any flow aid, sweetener, thinner, preservative, dyes/colorant, taste enhancer, surfactant, wetting agent, dispersant, disintegrant, suspension, stabilizer, isotonic agent, solvent or emulsifier licensed by the NMPA for use in humans or animals (e.g., livestock). For example, including but not limited to calcium carbonate, calcium phosphate, various sugars and starches, cellulose derivatives, gelatin, vegetable oils and polyethylene glycol.

The pharmaceutical composition described in the present invention may be formulated into solid, semi-solid, liquid or gaseous preparations, such as tablets, pills, capsules, powders, granules, ointments, emulsions, suspensions, solutions, suppositories, injections, inhalants, gels, microspheres and aerosols and the like.

The pharmaceutical composition described in the present invention may be manufactured by methods well known in the art, such as conventional mixing method, dissolution method, granulation method, sugar-coated pill method, grinding method, emulsification method, freeze-drying method, etc.

The compounds described in the present invention or the pharmaceutically acceptable routes of administration of salts thereof or pharmaceutical compositions thereof, including, but not limited to, oral, rectal, transmucosal, enteral administration, or topical, percutaneous, inhaled, parenteral, sublingual, intravaginal, intranasal, intraocular, intraperitoneal, intramuscular, subcutaneous, intravenous administration. The preferred route of administration is oral administration.

For oral administration, the pharmaceutical composition may be formulated by mixing the active compound with a pharmaceutically acceptable carrier well known in the art. These carriers enable the compounds of the present invention to be formulated into tablets, pills, lozenges, icings, capsules, liquids, gels, slurries, suspensions, etc., for oral administration to patients. For example, pharmaceutical compositions for oral administration, tablets may be obtained in the following manner: the active ingredient is combined with one or more solid carriers, if it is necessary to granulate the resulting mixture, and if a small amount of excipients need to be added to process into a mixture or granules to form tablets or tablet cores. The core can be combined with any coating material suitable for enteric-coated and processed into a coating formulation form that is more conducive to absorption by organisms (e.g., humans).

In summary, compared with the prior art, the beneficial effect of the present invention lies in:

The present invention models diabetic rats by streptozotocin (STZ), models the formation of diabetic nephropathy with a high-fat diet in rats, uses urine microalbumin (mALB) as an indicator to test whether the rat enters diabetic nephropathy, and uses rat body weight and blood glucose level as an indicator to observe whether the model is successful. The experimental results showed that the administration of diabetic rats with diabetic azoxide could delay the process of kidney damage in diabetic rats. Further, the same diabetic rat model was used to administer cromakalim, pinacidil, nicorandil, aprikalim, quinethazone, minoxidil, nicardipine KATP channel opener, from the detection results of urine microalbumin, there were different degrees of effect on reducing urinary microalbumin in rats with diabetic nephropathy.

### BRIEF DESCRIPTION OF THE DRAWINGS

FIG. 1 shows the effect of each experimental group of Example 2 on blood glucose.

FIG. 2 shows the effect of each experimental group on body weight of Example 2.

FIG. 3 shows the effect of each experimental group of Example 2 on mALB.

In FIG. 3, Group A was the blank control group, Group B was the negative control group, Group C was the dosing group administering 0.5 mg/kg diazoxide, and Group D was the dosing group administering 5 mg/kg diazoxide.

FIG. 4 shows the effect of different K_{ATP} channel opener experimental groups on mALB in Example 3.

FIG. 5 shows the effect of each experimental group of Example 2 on the glomeruli of rat kidney.

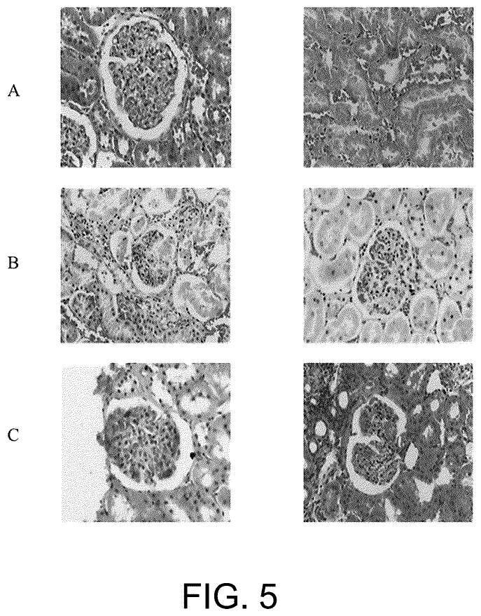

In FIG. 5, A) blank control group: 1) left: six-month rat, renal HE×100, glomerular volume increased significantly, glomerular cell proliferation; 2) Right: Experimental six-month rat, renal HE×400, renal tubular condition is good;

B) Negative control: 1) Left: Experimental six-month rat, renal HE×400, glomerular state is good, glomerular volume is significantly increased, tubular damage; Right: Six-month rat, renal HE×400, glomerulus significantly increased;

C) Medium-dose dosing group: 1) Left: six-month rat, renal HE×400, some glomeruli significantly increased in size, increased the number of cells, and some glomerular morphology was good; Right: Six-month rat, renal HE×400, glomerular volume increased, tubular vacuolar degeneration.

### DESCRIPTION OF THE EMBODIMENTS

The following are specific embodiments of the present invention, the technical solutions of the present invention are further described, but the scope of protection of the present invention is not limited to these embodiments. Any alteration or equivalent replacement that does not deviate from the idea of the present invention is covered by the scope of protection of the present invention.

In the present invention, the experimental instruments, experimental practice, experimental samples involved, the sources are as follows:

### 1. Animal Materials

Experiments were conducted in two groups at different times, with a total of 205 rats, of which 75 were used to study diazoxides and the other 130 were used for the study of other K_{ATP} channel agonists such as cromakalim, pinacidil, nicorandil, aprikalim, quinethazone, minoxidil, nicardipine.

205 male rats with 8-week SD (provided by the Animal Experiment Center of Zhejiang Academy of Medical Sciences) were selected with a weight of 200 g-250 g. Feeding conditions: room temperature is kept at about 25° C., humidity is about 60%, day and night are 12 h each, 24 h circulates ventilation, unless for special experiments (such as fasting to measure blood sugar, etc.). all animals eat freely and drink freely.

### 2. Main Drugs and Reagents of the Experiment

TABLE 1
Drugs and reagents

| Drug, reagent | manufacturer brand or manufacturer |
|---|---|
| Streptozotin (STZ) | MedChemExpress |
| Sodium hydroxide (NaOH) | Sinopharm Chemical Biotechnology |
| | Co., Ltd |
| Sodium hydroxymethylcellulose | Sinopharm Chemical Biotechnology |
| (CMC-Na) | Co., Ltd |
| Dilute hydrochloric acid (HCl) | Sinopharm Chemical Biotechnology |
| | Co., Ltd |
| High-fat feed | Nanjing Shengmin Scientific |
| | Research Animal Farm |
| Purified water | Wahaha |
| Litter wood chips | Zeya Technology |
| Absolute ethanol | Sinopharm Chemical Biotechnology |
| | Co., Ltd |
| Diazoxide | MedChemExpress |
| Cromakalim | MedChemExpress |
| Pinacidil | MedChemExpress |
| Nicorandil | Sigma |
| Aprikalim | MedChemExpress |
| Quinethazone | Sigma |
| Minoxidil | Sigma |
| Nicardipine | Sigma |
| Glucose | Sigma |
| ELISA kit (mALB, creatinine, | Wuhan Cloud Clone Technology |
| β2-MG, RBP4) | Co., Ltd |
| BUN kit | Nanjing JianCheng Biology |
| PAS staining kit | Solebor Biotechnology GmbH |
| HE staining | Solebor Biotechnology GmbH |
| Paraffin | Shanghai Yien Chemical |
| | Technology Co., Ltd |
| Xylene | Shanghai Yien Chemical |
| | Technology Co., Ltd |
| Neutral gum | Shanghai Suoqiao Biotechnology |
| | Co., Ltd |
| Immunohistochemical staining kit | Solebor Biotechnology GmbH |

### 3. Main Instruments and Equipment of the Experiment

TABLE 1
Instruments and equipment

| Instrument | Manufacturer/brand |
|---|---|
| Centrifuge | Beckman |
| Pure Water Meter | Nanjing Yipuida Technology |
| | Development Co., Ltd |
| Vertical automatic electric | Shanghai Shen'an Medical |
| pressure steam sterilizer | Equipment Factory |
| Blood glucose mete | Sinocare |
| Blood glucose test strip | Sinocare |
| Electronic Balance | Yingheng Electronic Balance Scales |
| Weight scale | Yingheng Electronic Balance Scales |
| No. 16 gavage needle | animal experiment mal |
| pH meter | Hangzhou LianCe Automation |
| | Technology Co., Ltd |
| Microplate reader | Beijing PuLang New Technology Co., |
| | Ltd |
| Constant temperature water bath | Yidu |
| Ultrasonic water bath | Jiangsu Shenglan Instrument |
| | Manufacturing Co., Ltd |
| Heparin lithium tube | Jiangxi Zhongjie Medical Equipment |
| | Co., Ltd |
| Coagulation tube | Jiangxi Zhongjie Medical Equipment |
| | Co., Ltd |
| Metabolic cage | Suzhou Guxiu Laboratory Animal |
| | Equipment Co., Ltd |
| Paraffin microtome | Leica |
| Light microscope | Leica |
| −80° C. ultra-low temperature | Sanyo Electric Corporation (Japan) |
| freezer | |
| Adjustable pipette gun | Eppendorf |
| 1 mL and 2.5 mL sterile | Shengguang Medical Products Co., Ltd |
| syringes | |
| Tissue embedding box | Jiangsu Shitai Experimental Equipment |
| | Co., Ltd |
| Adhesion slides | Jiangsu Shitai Experimental Equipment |
| | Co., Ltd |
| Coverslip | Jiangsu Shitai Experimental Equipment |
| | Co., Ltd |

### 4. Preparation of Main Experimental Reagents

### 1) Streptozotoxin (STZ)

500 mg of STZ is weighed out and mixed with 50 mL of sodium crate buffer (pH=4.5), a concentration of 0.1 mol/L, into a 50 mL centrifuge tube (outer tin foil) to prepare a solution at a concentration of 10 mg/ml. The solution was used within 10 minutes to prevent STZ failure.

### 2) 0.5% CMC-Na Solution

500 mg of CMC-Na is weighed out and mixed with 100 mL of ultrapure water into a 50 mL centrifuge tube to prepare a solution with a final concentration of 0.5%. The solution is shaken thoroughly in the ultrasonic machine where the temperature of the ultrasonic machine is set above 50° C. for convenience

### 3) Preparation of Sodium Hydroxide Solution

Step 1: Measure 80 mL of deionized water and place it in a plastic beaker (due to the large amount of exothermic heat during the NaOH dissolution process, do not use a glass beaker to avoid the glass beaker bursting).

Step 2: Weigh 20 g of NaOH and slowly add it to the beaker, stirring as it goes.

Step 3: After the NaOH is completely dissolved, set the volume to 100 mL with deionized water.

Step 4: Transfer the prepared solution to a plastic container and store it at room temperature.

### 4) Diazoxide

Weigh 1 g of diazoxide, add 6 mL of NaOH solution, and then add 450 mL of ultrapure water. and shake it well in the ultrasound machine until it is transparent. Add an appropriate amount of 5 μM HCl solution, adjust the pH to 7.5, and then set the volume to 500 mL. The final concentration of the solvent is of 5 mg/kg.

### 5) Glucose Solution

Weigh 25 g of glucose, add 45 mL of ultrapure water, shake the solvent thoroughly in the ultrasound machine and set the ultrasound machine temperature to 50° C. for easy dissolution. After dissolving thoroughly, set the volume to 50 ml. If used overnight, it must be placed in the refrigerator and stored at 4° C.

### Example 1 Establishment of Diabetic Nephropathy Model and Drug Detection Method

### 1. Establishment of Diabetes Model

STZ substantially and excessively damages islet-β cells through chemical toxicity (Saini K, Thompson C, Winterford C M, Walker N I, Cameron D P. Streptozotocin at low doses induces apoptosis and at high doses causes necrosis in a murine pancreatic b cell line, INS1[J]. International Union of Biochemistry and Molecular Biology Life. 1996, 39(6): 1229-1236). The pathophysiological characteristics of STZ modeled rats are relatively close to that of humans. Their blood can be collected repeatedly to monitor index changes and enough kidney tissues can be obtained for subsequent tissue section analysis. Therefore, STZ diabetic rat models are used. In addition, due to the high toxicity of STZ, multiple injections at low doses can establish better models. Keep feeding a high-fat diet daily after modeling until the end of the experiment. A high-fat diet can cause diabetic nephropathy in STZ rats.

The specific modeling method is described in Example 2.

### 2. Establishment of Diabetic Nephropathy Model and Establishment of Measurement Indicators

Urine microalbumin (mALB) is an early diagnostic index of kidney injury, and mALB was used as an indicator to test whether rats entered diabetic nephropathy, and rat weight and blood glucose levels were used to observe whether the model was successful.

mALB is one of the most important monitoring indicators of nephropathy. Clinically, mALB >20 μg/min is nephropathy and indicating that there is kidney damage. Since the measurement results of mALB in humans and mice are different. The difference between the dosing group (group C) and the negative control group (group B) is used to judge whether the test results of this experiment are valid. The normal mALB value in this experiment was only used as a reference value, which is using the data of group A (blank control group).

The changes in the glomerulus mainly include the expansion of the mesangial stroma, any degree of arteriole hyalasia, basement membrane thickening and interstitial fibrosis.

### 3. Collection of Blood and Urine Samples and Retention of Tissue Samples

At the beginning of the experiment, urine and blood of all rats were collected for the detection of the required indicators, and the initial indicators were used as a timely reference for the disease progression of the rats. After the start of the experiment, all rats were sampled with the Elisa kit every other month to detect mALB and other data.

Rat urine collection: urine is collected by metabolic cage, urine is collected after 24 h, the total amount of urine is recorded, and the urine output at 24 h is calculated. Take 1 ml of urine, centrifuge and take the supernatant and store at −80° C. for testing.

Rat blood collection: 3 mL of rat blood was collected by tail clipping, the blood was collected in a medical blood test tube containing a procoagulant, left at room temperature for 2 h or 4° C. overnight, centrifuged at 1000 RCF for 20 minutes, the supernatant was taken and stored at −80° C. for testing. Alternatively, collect 3 mL of rat blood by tail clipping, blood collected in a test tube of heparin lithium, centrifuge at 1,000 RCF for 20 min, take the supernatant, and store at −80° C. for testing.

### 4. Detection Method

All rat blood and urine were collected during molding, and the Elisa kit was sampled to detect mALB and other biochemical indexes. All the above indicators were tested every other month after the start of the experiment until most of the mice had diabetic nephropathy.

A 24 h urine sample was collected from rats for testing, and the collected sample was centrifuged at 15 min, centrifuged at 4° C. at 1000 ref for 20 min, and the mALB value was detected by supernatant or stored at −20° C.

Before starting the experiment, you need to do the following:
(1) Place all reagents (including specimens) in the room and equilibrate to room temperature (18-25° C.) before use.
(2) The standard is diluted to the corresponding gradient. Take a bottle of standard, add 1 mL of standard diluent (at this time the concentration is 1000 μg/mL), mix gently, let stand at room temperature for 10 minutes, and gently shake every 2 minutes to mix. After mixing, dilute it to 100 μg/mL, take 4 EP tubes (add 600 μL of standard dilute to each EP tube), triple dilute the standard from a concentration of 100 μg/mL to 33.33 μg/mL, 11.11 μg/mL, 3.70 μg/mL, 1.23 μg/mL, and use the standard dilution as a blank well (0 μg/mL).
(3) Working solution of the detection solution A: add 150 μL of reagent dilution to the mother liquor of the detection solution A, stand at room temperature for 10 minutes, and shake gently every 2 minutes to have a well-mix. Before use, dilute with the detection diluent A at 1:100, and mix the solution well with the shaker. Calculate the required amount at 50 μL/well for dilution before use (0.1-0.2 mL more needs to be prepared).
(4) Working solution of the detection solution B: Detection solution B needs to be centrifuged at 5000 ref for 10 s before each use to deposition the liquid from the tube wall or cap to the bottom of the tube. Immediately before use, dilute with the detection diluent B at 1:100, and mix them well with the shaker. Calculate the required amount of 100 μL/well before use for dilution (0.1-0.2 mL more required).
(5) Concentrated washing solution: 30-fold dilution of the concentrated washing solution (the dilution amount is calculated according to your own sample size).

After all the supplies are ready, perform the experimental operation as follows:
(1) Start sampling after setting the standard wells, sample wells to be tested, and blank wells. Set 5 wells of the standard wells and add 50 μL of the Standard with different concentrations sequentially (you can also set 10 wells, set two wells for each standard, and finally take the average). Add 50 μL of diluted Standard to the blank well, and 50 μL of the sample to be tested from the well to be tested (mark the sample to avoid later confusion). Immediately afterwards, add 50 μL of detection solution A working solution per well, vibrate gently, mix well, be careful not to have bubbles, add a coating to the enzyme plate (do not touch the bottom of the enzyme plate to avoid inaccurate light absorption), and incubate at 37° C. for 1 hour.
(2) Washing by the washing solution. After incubation is complete, discard the liquid in the wells (which can be aspirated with a pipette or by tapping on absorbent paper), wash each well with 350 μL of washing solution, soak for 1-2 minutes, and gently pat the plate on absorbent paper to remove all liquid from the wells. Repeat the above three times (the last wash should be aspirated or poured out of all remaining wash buffer with a pipette).
(3) Add 100 μL working solution of the detection solution B to each well, add a coating to the enzyme plate (do not touch the bottom of the enzyme plate to avoid inaccurate absorption), and incubate at 37° C. for 30 minutes.
(4) Aspirate with the pipette gun or pat on the absorbent paper to dry and repeat the plate washing 5 times (the operation steps are the same as step 2).
(5) Add 90 μL substrate solution per well, enzyme plate plus coating, 37° C. protected from light color development (reaction time is controlled at 10-20 minutes, not more than 30 minutes (when the last three concentrations of standard wells have obvious gradient blue, the first 3 wells gradient is not obvious, it can be terminated).
(6) Add 50 μL of stop solution per well to terminate the reaction (blue turns yellow immediately).
(7) After ensuring that there are no water droplets at the bottom of the microplate plate and no bubbles in the wells, immediately measure the optical density (OD value) of each well with a microplate reader at a wavelength of 450 nm.
(8) According to the OD value of each standard and sample, deduct the OD value of the blank hole and make a graph (if the complex hole is set, the average value should be calculated), draw the standard curve, substitute the OD value of the sample into the equation, and calculate the sample concentration.

### Example 2 Study the Effect of Diazoxide on Diabetic Nephropathy

### [Purpose of the Experiment]

Based on the rat model and detection method of diabetic nephropathy constructed in Example 1, the present embodiment aims to investigate whether high, medium and low doses of diazoxide have a therapeutic effect on diabetic nephropathy.

### [Experimental Method]

After 7 days of adaptive rearing of SD rats, 10 of them were randomly selected as a blank control group (group A), i.e., without STZ injection. The remaining 65 rats were randomly divided into 4 groups, of which 14 were negative control groups, and the remaining 17 in each group After 2 weeks of high-fat feed feeding to induce insulin resistance, Rats began to establish a diabetes mode. The specific modeling method is as follows:
(1) After fasting for at least 12 h, Rats were injected STZ at a dose of 30 mg/kg. They were allowed to eat and drink freely after injection;
(2) Measure fasting blood glucose after STZ injection for 48 h. It would be considered as a successful model if a rat had its blood glucose value higher than 16.7 mmol/L;
(3) For rats whose blood glucose values did not reach the standard, they continued to inject STZ at another dose of 30 mg/kg after fasting for 12 h until the blood glucose met the requirements.
(4) All rats that meet the requirements to test whether the blood glucose still meets the standard after one week. If the blood glucose level dropped to less that the standard, the rats would be repeated the above procession. At this time, the rats that successfully modeled had a significant increase in urine output and the symptoms of diabetes appeared.

All diabetic rats who met the criteria were randomly divided into 4 groups: Group B was rats injected with STZ only, as a negative control; Group C received gavage of diazoxide at a dose of 0.5 mg/kg per day (low-dose group); Group D received gavage of 5 mg/kg of diazoxide per day (medium-dose group); Group E received gavage of 50 mg/kg of diazoxide per day (high-dose group). Due to the large body surface area of rats, the doses of groups C, D and E were equivalent to 0.095 mg/kg, 0.95 mg/kg, and 9.5 mg/kg in humans (according to the literature: Anroop B Nair, Shery Jacob. A simple practice guide for dose conversion between animals and human. Journal of Basic and Clinical Pharmacy. 2016, 7(2):27-31.), with 95% binding capacity to albumin, similar to humans. All groups of rats were fed high-fat feed.

### [Experimental Results]

The data obtained in the experiment were recorded and processed by Micorosoft Office Excel software, and the P value was calculated, and whether there was a significant difference with P<0.05.

The experimental results showed that after half a month (2 weeks) of STZ injection, the urine of rats in each experimental group increased significantly. The average urine output of rats in the blank control group (group A, the same below) was 6.50±0.52 mL at 24 hours, and the average urine output in 24 hours in the STZ control group (group B, the same below) and rats in the dosing group (including group C, D and E) was 27.23±3.82 mL, which was a significant difference compared with the blank control group (p<0.01).

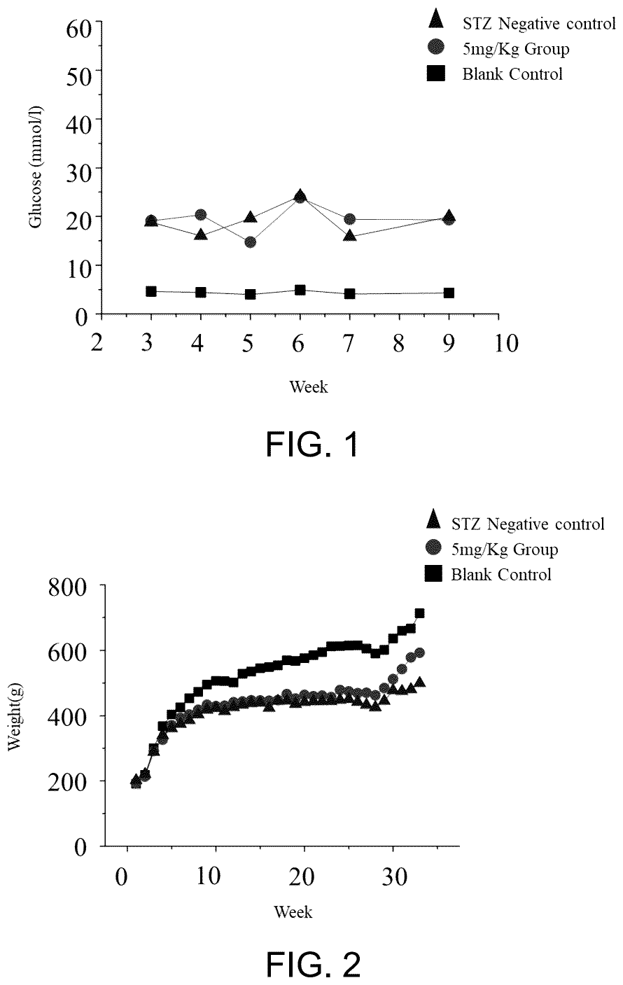

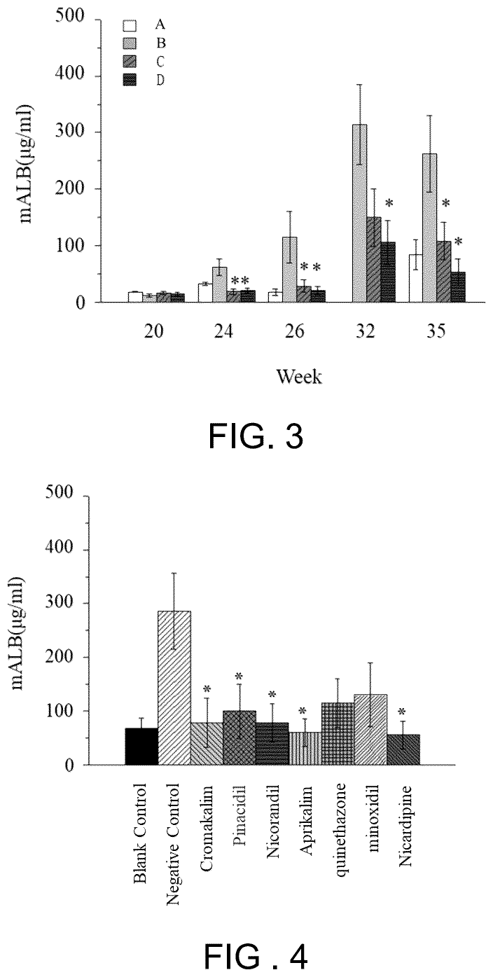

In addition, rats in the high-dose E group all died before diabetic nephropathy was modeled, of which 13 died in the first four months and the other 5 died in the fifth and sixth months. It was speculated that the death may be related to the high blood glucose elevation of diabetic rats. Therefore, the statistics of Table 1, FIG. 1, FIG. 2, FIG. 3 and FIG. 5 are not included those rats in group E.

As shown in FIG. 1, observing the blood glucose data in weeks 3-9, the average blood glucose of the blank control group (▪) was between 4.0-4.9 mmol/L, while the average blood glucose of the STZ control group (▴) and the 5 mg/kg dosing group (●) was basically maintained between 18-19 mmol/L, which was higher than the 16.7 mmol/L specified in the literature (see step 2 of the preceding modeling method). Moreover, rats in the STZ control group and the 5 mg/kg dosing group already had the symptoms of diabetes, as well as symptoms such as irritability, slow response, dry and yellow hair color, indicating that the modeling of the diabetes model was successful.

As shown in FIG. 2, the rat weight trend plot is obtained: blank control group (▪)>STZ control group (▴)=5 mg/kg dosing group (●). The weight of the blank control group showed a stable increase due to long-term consumption of high-fat feed and no drug effect (including STZ) and remained as the heaviest group. Due to STZ injury (the mean blood glucose value of the group was stable above 16.7 mmol/L), the weight of the STZ control group and the 5 mg/kg dosing group remained low (much lower than that of the blank control group), and there was no statistically significant difference body weight between the STZ control group and the 5 mg/kg dosing group (P>0.05) before the treatment. However, there were significant differences in body weight between these two groups and the no treatment group (blank control group, p<0.01).

As shown in Table 1 and FIG. 3, analyzing the mALB data shows:
(1) At week 24th of the experiment, the mALB value of rats was randomly sampled, and it was found that the urine microalbumin (mALB) of group B was 61.9±14.7 μg/mL, which was about 6 times the mALB value at week 20th; The mALB values in group C were 18.2±4.7 μg/mL, and the mALB values in group D were 19.8±4.3 μg/mL, both of which were comparable to the mALB values at week 20th. It showed that the rats in group B had developed kidney damage at week 24th, while the rats in group C and group D had not yet developed symptoms of diabetic nephropathy.

Compared with group B, there were significant differences (p<0.05) in group C and D in the dosing group, indicating that diazoxide can delay the process of kidney damage in diabetic rats and play a preventive or therapeutic role in patients with diabetic nephropathy in the early stage.
(2) At the 26th week of the experiment, the mALB values of rats were also randomly sampled, and it was found that compared with group A, the mALB values of group B showed a very significant difference (p<0.05), but there was no significant difference between groups C and D (p>0.05).

Compared with group B, there was a significant difference between group C and group D (p<0.05), indicating that both low- and medium-dose diazoxide still had anti-microproteinuria and delayed diabetic symptoms.
(3) At week 32th of the experiment, the mALB value of the rats was also observed, and the mALB value of group B was very high, indicating that the rats in group B had completely entered diabetic nephropathy.

Compared with Group B, the mALB values of Group C and Group D decreased to a certain extent, with significant differences in Group D (p<0.05) and no significant differences in Group C (p>0.05).
(4) Before the end of the experiment, the sampling results of all remaining rats were shown in Table 1, and compared with group B, there was still a significant difference in the medium-dose D group (p<0.05), and the low-dose C group had a certain degree of reduction at 32 weeks and 35 weeks, but it was close to but did not reach a significant difference (p<0.05). In addition, group B showed an increase in compensatory glomerular filtration rate, while the dosing group (group C, group D) could reduce the increase of glomerular filtration rate.
(5) Experiments have shown that diazepine at a dose of 0.5 mg/kg in administration can delay the progression of diabetic nephropathy and have a sustained protective effect on the kidneys of diabetic rats.

TABLE 1
Urine microalbumin value at different times (mean ± SE) of each experimental group

| | mALB (μg/ml) | | | | |
|---|---|---|---|---|---|
| Groups | 20 Week | 24 Week | 26 Week | 32 Week | 35 Week |
| A (Blank Control) | 18.0 ± 0.6 | 32.1 ± 3.6 | 16.7 ± 5.9 | NA | 83.7 ± 26.7 |
| B (Negative Control) | 11.9 ± 3.2 | 61.9 ± 14.7# | 114.4 ± 45.7# | 313.4 ± 70.7 | 262.3 ± 67.6# |
| C (0.5 mg/Kg Diazoxide) | 15.6 ± 2.9 | 18.2 ± 4.7* | 28.4 ± 10.8* | 149.5 ± 50.3 | 108.0 ± 33.7 |
| D (5 mg/Kg Diazoxide) | 14.5 ± 3.0 | 19.8 ± 4.3* | 19.9 ± 7.4* | 105.5 ± 38.9* | 52.8 ± 23.4* |

### [Note]

a: #p<0.05, ##p<0.01 compared to group A; *p<0.05, **p<0.01 compared to group B; b: “NA” means untested.

In addition, clinically patients with nephropathy will have an increase in glomerular volume and a higher number of cells in the early stage, and with the progression of the disease, there will be widening of the mesangial area, the mesangial cells and mesangial stroma will increase, and the tubular epithelial cells will have cell edema, that is, often called granular degeneration or vacuolar degeneration, tubulointerstitial fibrosis and other symptoms. In this experiment, the glomerular volume changes of rat kidney sections were observed by HE staining, and the results of renal staining sections in each experimental group are shown in FIG. 5.

As shown in FIG. 5, all model mice had significant glomerular enlargement and cell increase, indicating that the model of diabetic nephropathy was successfully modeled.

In addition, from FIG. 5, it can be seen that:
(1) The glomerular volume of group A (blank control group) increased slightly, but its proximal and distal convoluted tubules were in good condition.
(2) Group B (negative control group) had significant increase in glomeruli and tubular damage.
(3) In group D, taking the dose of 5 mg/kg of diazoxide as an example, part of the tubular vacuolar degeneration in group D (see the right figure of group D in FIG. 5), and tubular lesions occurred in group B and group D.

### Example 3 Study of the Effect of Other K_{ATP} Agonists on Diabetic Nephropathy

### [Purpose of the Experiment]

On the basis of the rat model and detection method of diabetic nephropathy constructed in Example 1, the present embodiment aims to investigate whether the remaining KATP agonists other than diazepine have a therapeutic effect on diabetic nephropathy.

### [Experimental Method]

After 130 SD rats were recuperated adaptively for 7 days, 10 were randomly selected as a blank control group, i.e., without STZ injection. The remaining 120 rats were randomly divided into 8 groups (including: negative control group, 1 mg/kg Cromakalim group, 1 mg/kg Pinacidil group, 0.5 mg/kg Nicorandil group, 1 mg/kg Aprikalim group, 1 mg/kg quinethazone group, 0.5 mg/kg minoxidil group, 5 mg/kg nicardipine group), 15 in each group, after inducing insulin resistance after 2 weeks of high-fat feeding, the diabetes model was established, and the specific modeling method and detection method were the same as Example 2.

### [Experimental Results]

The data obtained in the experiment were recorded and processed by Micorosoft Office Cle software, and the P value was calculated, and whether there was a significant difference with P<0.05. The results of mALB values at week 30 of the experiment are shown in Table 2 and FIG. 4.

TABLE 2
Urine microalbumin value of each K_{ATP} agonist (mean ± SE)

| | mALB (μg/ml) | | | | | | | | |
|---|---|---|---|---|---|---|---|---|---|
| Groups | Blank Control | Negative Control | Cromakalim | Pinacidil | Nicorandil | Aprikalim | quinethazone | minoxidil | nicardipine |
| Result | 67.3 ± 19.7* | 285.4 ± 70.8* | 77.6 ± 45.6* | 99.7 ± 50.7* | 78.2 ± 35.1* | 59.7 ± 25.4* | 114.6 ± 45.3 | 130.4 ± 60.3 | 55.4 ± 25.6* |

Note: Compared with the negative control group, *p<0.05, **p<0.01.

From Table 2 and FIG. 4, it can be seen that compared with the negative control group, K_{ATP} channel agonists Cromakalim, Pinacidil, Nicorandil, Aprikalim and Nicardipine have an average significant effect on reducing the amount of microalbumin in diabetic nephropathy urine (p<0.05).

1. Application of potassium ATP channel modulators in the preparation of anti-diabetic nephropathy drugs.

2. The application according to claim 1, wherein the diabetic nephropathy is type 1 diabetes mellitus and/or type 2 diabetes mellitus complicated by diabetic nephropathy, preferably in stage I, II. or III diabetic nephropathy.

3. The application according to claim 1, wherein the potassium ATP channel regulator comprises a potassium ATP channel opener or a potassium ATP channel inhibitor.

4. The application according to claim 3, wherein the potassium ATP channel regulator is selected from one of diazoxide, cromakalim, pinacidil, nicorandil, aprikalim, quinethazone, minoxidil, nicardipine.

5. The application according to claim 3, wherein the potassium ATP channel opener is diazoxide administered at a dose of 0.5-5 mg/kg.

6. A pharmaceutical composition for the prevention or treatment of diabetic nephropathy, comprising any one of the right 1-5 potassium ATP channel modulators as the active ingredient.

7. The pharmaceutical composition according to claim 6, wherein it further comprises pharmaceutically acceptable excipients.

8. The pharmaceutical composition according to claim 6, wherein the pharmaceutical composition is used for the prevention or treatment of early diabetic nephropathy, comprising stage I, II or III in the evolution of diabetic nephropathy stage.

9. The pharmaceutical composition according to claim 6, wherein the dosage form of the pharmaceutical composition is selected from one of tablets, capsules, granules, injections, patches, gels.

10. The pharmaceutical composition according to claim 6, wherein the pharmaceutically acceptable excipients are fillers, disintegrants, binders, thinners, lubricants, conditioners, solubilizers, co-solvents, emulsifiers one or more of them.
